(19) United States
(12) Patent Application Publication
(10) Pub. No.: US 2006/0056589 A1
(43) Pub. Date: Mar. 16, 2006

(54) Radiation-induced cellular adaptive response

(75) Inventor: Bevin P. Engelward, Jamaica Plain, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Massachusetts Institute of Technology, Cambridge, MA (US)

(21) Appl. No.: 11/210,252

(22) Filed: Aug. 23, 2005

Related U.S. Application Data
(60) Provisional application No. 60/605,856, filed on Aug. 31, 2004.
(60) Provisional application No. 60/624,747, filed on Nov. 3, 2004.

(51) Int. Cl. A61N 5/10

(52) U.S. Cl. 378/65

(57) ABSTRACT

One aspect of the present invention relates to a method for determining an adaptive response of a tumor during radiation therapy. A second aspect of the present invention relates to a method for determining a substantially optimal dose of radiation therapy based on a cells ability to undergo an adaptive response. Another aspect of the present invention relates to a method for identifying small molecule compounds that are effective chemotherapeutic agents for use during and after radiation therapy.

### RELATED APPLICATIONS

This application claims priority to U.S. Provisional Patent Application Ser. Nos. 60/605,856, filed on Aug. 31, 2004; and 60/624,747, filed on Nov. 3, 2004; both applications are hereby incorporated by reference in their entirety.

### GOVERNMENT SUPPORT

The invention was made with support provided by the National Institutes of Health (CA84740); therefore, the government has certain rights in the invention.

### BACKGROUND OF THE INVENTION

Cancer is primarily treated with one or a combination of three types of therapies: surgery, radiation, and chemotherapy. Surgery, which involves the bulk removal of diseased tissue, can be effective in removing tumors located at certain sites, for example, in the breast, colon, and skin; however, it cannot be used in the treatment of tumors located in inaccessible areas, nor in the treatment of disseminated neoplastic conditions, such as leukemia. Radiation therapy and/or chemotherapy are thus frequently combined with surgery and are often the primary course of treatment for numerous cancers.

Radiation therapy is based on the principle that high-dose radiation delivered to a target area will preferentially kill dividing cells, and thus be more toxic to rapidly dividing tumor cells than to normal cells. Chemotherapy is based on the use of agents or drugs that injure or kill cells by affecting any number of cellular mechanisms. Both radiation therapy and chemotherapy are typically administered in multiple doses over a period of weeks to months depending on the type and stage of the cancer. The successful use of radiation therapy and chemotherapeutic agents to treat cancer depends upon the differential killing of cancer cells compared to its side effects on critical normal tissues.

Finding the right combination of chemotherapeutic drugs and/or radiation therapy is determined empirically by identifying courses of treatment that appear to be effective for a population of patients. Based on these population studies, a course of radiation treatment is decided for an individual patient prior to starting treatment and that course of therapy is maintained unless the patient cannot withstand the toxicity of treatment. As a result, individual patients frequently endure adverse side effects arising from both treatments. Nausea, vomiting, and fatigue are the most common and severe side effects, but a patient may also experience alopecia (hair loss), cytopenia, infection, cachexia, or mucositis, as well as neurological, pulmonary, cardiac, reproductive and/or endocrine complications. An additional complication to cancer treatment is that patients may also become resistant to repeated treatment approaches.

More than half of all cancer patients today receive some form of radiation therapy. Radiation-induced and chemotherapy-induced side effects significantly impact the quality of life of the patient and may dramatically influence patient compliance with treatment. Minimizing the adverse side effects and increasing the effectiveness of treatment are crucial to the clinical management of cancer patients. Thus, there remains a need for improved therapeutic methods using radiation and chemotherapy to treat cancer.

### SUMMARY OF THE INVENTION

One aspect of the present invention relates to a method for assessing an adaptive response of a tumor exposed to chronic radiation therapy, comprising the steps of administering a course of radiation therapy to a subject prior to surgery to remove a tumor; surgically removing the tumor; and monitoring the tumor for an adaptive response. In certain embodiments, the present invention relates to the aforementioned method, wherein the subject is exposed to a total dose of radiation from about 5 to 15 Gy prior to surgery to remove a tumor. In certain embodiments, the present invention relates to the aforementioned method, wherein the total dose of radiation is administered in five doses. In certain embodiments, the present invention relates to the aforementioned method, wherein each dose of radiation about 1 to 3 Gy. In certain embodiments, the present invention relates to the aforementioned method, wherein the source of radiation is selected from the group consisting of x-ray radiation, gamma-ray radiation, UV radiation, microwaves, electronic emissions, and particulate radiation. In certain embodiments, the present invention relates to the aforementioned method, wherein the source of radiation is x-ray radiation. In certain embodiments, the present invention relates to the aforementioned method, further comprising obtaining a healthy tissue sample from a subject during surgery to remove a tumor mass, and monitoring said healthy tissue for an adaptive response. In certain embodiments, the present invention relates to the aforementioned method, wherein the adaptive response is monitored by measuring the expression of γ-H2A expression. In certain embodiments, the present invention relates to the aforementioned method, wherein an adaptive response is monitored by measuring cell survival.

A second aspect of the invention relates to a method for assessing an adaptive response of a tumor exposed to chronic radiation therapy, comprising the steps of obtaining a tumor tissue sample from a subject; exposing the tumor tissue sample to radiation ex vivo; and monitoring the tumor tissue sample for an adaptive response. In certain embodiments, the present invention relates to the aforementioned method, further comprising obtaining a healthy tissue sample from a subject; exposing said healthy tissue sample to radiation ex vivo; and monitoring said healthy tissue for an adaptive response. In certain embodiments, the present invention relates to the aforementioned method, wherein the tissue sample is obtained from a subject during a biopsy procedure. In certain embodiments, the present invention relates to the aforementioned method, wherein the tissue sample is obtained during surgery. In certain embodiments, the present invention relates to the aforementioned method, wherein the tissue sample is exposed to a dose of radiation from about 1 to 3 Gy. In certain embodiments, the present invention relates to the aforementioned method, wherein the tissue sample is exposed to four or five doses of radiation. In certain embodiments, the present invention relates to the aforementioned method, wherein the source of radiation is selected from the group consisting of x-ray radiation, gamma-ray radiation, UV-irradiation, microwaves, electronic emissions, and particulate radiation. In certain embodiments, the present invention relates to the aforementioned method, wherein the source of radiation is x-ray radiation. In certain embodiments, the present invention relates to the aforementioned method, wherein the adaptive response is monitored by measuring γ-H2A expression. In certain embodiments, the present invention relates to the aforementioned method, wherein an adaptive response is monitored by measuring cell survival.

A third aspect of the present invention relates to a method for determining a substantially optimal dose of radiation needed to inhibit tumor growth, comprising the steps of administering a course of radiation therapy to a subject prior to surgery to remove a tumor; removing the tumor by surgery; dividing the tumor into a plurality of samples; exposing independently the plurality of samples to subsequent doses of radiation; and monitoring the plurality of samples for adaptive responses. In certain embodiments, the present invention relates to the aforementioned method, wherein the subject is exposed to a total dose of radiation from about 5 to 15 Gy prior to surgery to remove a tumor. In certain embodiments, the present invention relates to the aforementioned method, wherein the total dose of radiation is administered in five doses. In certain embodiments, the present invention relates to the aforementioned method, wherein each dose of radiation is about 1 to 3 Gy. In certain embodiments, the present invention relates to the aforementioned method, wherein the source of radiation is selected from the group consisting of x-ray radiation, gamma-ray radiation, UV radiation, microwaves, electronic emissions, and particulate radiation. In certain embodiments, the present invention relates to the aforementioned method, wherein the source of radiation is x-ray radiation. In certain embodiments, the present invention relates to the aforementioned method, further comprising obtaining a healthy tissue sample from a subject during surgery to remove a tumor mass, and monitoring said healthy tissue for an adaptive response. In certain embodiments, the present invention relates to the aforementioned method, wherein the sample is exposed to subsequent doses of radiation varying from about 0.5 to about 4 Gy. In certain embodiments, the present invention relates to the aforementioned method, wherein the sample is exposed to four or five doses of radiation. In certain embodiments, the present invention relates to the aforementioned method, wherein the source of radiation for subsequent doses of radiation is selected from the group consisting of x-ray radiation, gamma-ray radiation, UV radiation, microwaves, electronic emissions, and particulate radiation. In certain embodiments, the present invention relates to the aforementioned method, wherein the source of radiation is x-ray radiation. In certain embodiments, the present invention relates to the aforementioned method, wherein the adaptive response is monitored by measuring the expression of γ-H2A expression. In certain embodiments, the present invention relates to the aforementioned method, wherein an adaptive response is monitored by measuring cell survival.

Yet another aspect of the present invention relates to a method for determining a substantially optimal dose of radiation needed to inhibit tumor growth, comprising the steps of obtaining a tumor tissue sample from a subject; exposing the tumor tissue sample to varying doses of radiation ex vivo; and monitoring the adaptive response of the tumor tissue sample. In certain embodiments, the present invention relates to the aforementioned method, further comprising obtaining a healthy tissue sample from a subject; exposing said healthy tissue sample to radiation ex vivo; and monitoring said healthy tissue for an adaptive response. In certain embodiments, the present invention relates to the aforementioned method, wherein the tissue sample is obtained from a subject during a biopsy procedure. In certain embodiments, the present invention relates to the aforementioned method, wherein the tissue sample is obtained from a subject during surgery. In certain embodiments, the present invention relates to the aforementioned method, wherein the tissue sample is exposed to varying doses of radiation range from about 0.5 to about 4 Gy. In certain embodiments, the present invention relates to the aforementioned method, wherein the tissue sample is exposed to four or five doses of radiation. In certain embodiments, the present invention relates to the aforementioned method, wherein the source of radiation is selected from the group consisting of x-ray radiation, gamma-ray radiation, UV-irradiation, microwaves, electronic emissions, and particulate radiation. In certain embodiments, the present invention relates to the aforementioned method, wherein the source of radiation is x-ray radiation. In certain embodiments, the present invention relates to the aforementioned method, wherein the adaptive response is monitored by measuring γ-H2A expression. In certain embodiments, the present invention relates to the aforementioned method, wherein an adaptive response is monitored by measuring cell survival.

The present invention also relates to a method for identifying chemotherapeutic drugs that are effective during and after radiation therapy, comprising the steps of pre-adapting target cells to radiation; screening the pre-adapted target cells against a plurality of small molecule compounds; and identifying small molecule compounds that induce DNA damage in the pre-adapted target cells. In certain embodiments, the present invention relates to the aforementioned method, wherein the target cells are pre-adapted to radiation following exposure to about 1 to about 3 Gy of radiation. In certain embodiments, the present invention relates to the aforementioned method, wherein the target cells are pre-adapted to radiation following exposure to about four or about five doses of radiation. In certain embodiments, the present invention relates to the aforementioned method, wherein the source of radiation to pre-adapt the target cells is selected from the group consisting of x-rays, gamma-rays, UV-irradiation, microwaves, electronic emissions, and particulate radiation. In certain embodiments, the present invention relates to the aforementioned method, wherein the source of radiation to pre-adapt the target cells is x-ray radiation. In certain embodiments, the present invention relates to the aforementioned method, wherein small molecule compounds that induce DNA damage in the pre-adapted target cells are identified by monitoring cell survival. In certain embodiments, the present invention relates to the aforementioned method, wherein small molecule compounds that induce DNA damage in the pre-treated target cells are identified by monitoring the induction of cell survival. In certain embodiments, the present invention relates to the aforementioned method, wherein small molecule compounds that induce DNA damage in the pre-adapted target cells are identified by detecting the expression of γ-H2A.

### BRIEF DESCRIPTION OF THE FIGURES

FIG. 1 shows radiation induced homologous recombination in FYDR mice. a, Arrangement of the FYDR recombination substrate. Large arrows represent expression cassettes, yellow regions indicate coding sequences and black boxes indicate the positions of deleted sequences. Note that deletions sizes are not to scale. An image of a recombinant fluorescent FYDR cell is shown on the far right. b, Treatment conditions for three mouse cohorts. Animals in the control cohort were sham treated, those in the acute cohort were exposed to 7.56 Gy at 28 days of age, and those in the chronically irradiated cohort were exposed daily to 28 cGy from 7 to 33 days of age. DOB indicates date of birth. c, Recombinant cell frequencies in cutaneous tissues of control, acute and chronically irradiated animals. d, Recombination rates of mouse adult fibroblasts (MAFs) isolated from mice in the control and chronically irradiated cohorts (calculated by the p₀ method (Luria and Delbruck (1943) Genetics 28:491-511)). Due to the toxicity associated with acute exposure to 7.56 Gy, MAFs could not be cultured from mice in the acute cohort. Mean±SE for 3-4 independent experiments is shown. e, Calculated recombination rates in vivo based on the p₀ method (Luria and Delbruck (1943) Genetics 28:491-511). The recombination rate among acutely exposed animals has been omitted, since the toxicity associated with the acute exposure impinges on the number of cell divisions post irradiation to an unknown extent. The 95% confidence limits were calculated using standard statistical methods, given that p₀ can be considered a binomial parameter (Lea and Coulson (1949) J. Genetics 49:264-285). Asterisk indicates p<0.05; standard binomial parameter statistics (Roche and Foster (2000) Methods 20:4-17).

FIGS. 2a-c are graphs showing the effects of radiation on gene expression and methylation in vivo. a, Levels of RNA transcripts expressed from the FYDR recombination substrate as measured by real-time PCR. Averages relative to GAPDH are shown. b, Lysates from cutaneous tissue were immunoblotted using antibodies against Ku70, Polβ, Ape1 and GAPDH as a loading control. Protein levels relative to those of control animals are shown. Representative western blots from among 8 independent experiments are shown. c, Global genome methylation level as measured by cytosine extension; values relative to control animals are shown. a-c, Mean±SE is shown; asterisks indicate p<0.05, Student's T-test.

FIGS. 3a-c are graphs showing chronic irradiation suppresses recombination in cultured cells. a, Recombinant cell frequencies and recombination rates for control and chronically irradiated MAFs. Each MAF culture was derived from ear tissue of an independent mouse. b, Recombinant cell frequencies and recombination rates for control and chronically irradiated mouse embryonic fibroblasts (MEFs) derived from a FYDR embryo. a-b, Cells were exposed in culture to 28 cGy/day or sham treated for six days. Scatter plots show recombinant cell frequencies with horizontal bars indicating median frequencies. Histograms show the recombination rates±SE (rates were calculated using the MSS Maximum Likelihood Method and SE was calculated as previously described (Roche and Foster (2000) Methods 20:4-17). c, SCE frequencies in MAFs exposed to 28 cGy/day for four days. Mean±SD among 18-20 independent metaphase spreads are shown. a-c, Asterisks indicate p<0.05, Student's T-test.

FIGS. 4a-d are photographs showing that rare recombinant cells can be seen within intact pancreatic tissue of FYDR mice. a) Freshly excised pancreatic tissue was stained with Hoechst and imaged under 10×. Recombinant cells appear yellow amidst a sea of non-recombinant cells. Recombinant cells divide to give rise to fluorescent daughter cells, yielding the appearance of singles, doublets, quadruplets etc. 60×. b) Fluorescent cells can be see in frozen sections. H&E staining of the adjacent section reveals the exact cells that carry recombined DNA. c) In young mice, the foci of recombinant cells generally contain a few cells, and rarely occur in patches. In older mice, most recombinant foci are much larger, indicating that recombinant cells have clonally expanded as the animal aged. d) One of the older FYDR mice had a spontaneous pancreatic tumor. The pattern of fluorescence in the normal pancreatic tissue of this mouse had highly irregular patterns of fluorescence. Most of the mouse's cells appeared to be fluorescent within the tumor itself.

### DETAILED DESCRIPTION OF THE INVENTION

Overview

In one aspect, the invention relates to a method for discerning an adaptive response of a tumor during chronic radiation therapy. Determining the ability of a tumor tissue sample obtained from a subject to undergo an adaptive response may be predictive of a subject's ability to respond effectively to radiation and/or chemotherapy. In certain embodiments, an adaptive response may be determined in a subject following the administration of several doses of radiation prior to removing a tumor by surgery, removing the tumor by surgery or tumor tissue by biopsy, and monitoring the tumor for an adaptive response. In an alternate embodiment, a tumor tissue sample may be obtained from a subject during a biopsy procedure or during surgery to remove a tumor mass and the ability of the tumor to undergo an adaptive response may be determined entirely ex vivo.

In a second aspect, the invention discloses a method for determining a substantially optimal dose of radiation needed to inhibit and/or eradicate tumor growth in a subject. In certain embodiments, a substantially optimal dose of radiation may be determined by administering to a subject several doses of radiation therapy prior to removing the tumor by surgery, removing the tumor by surgery, and assessing the response of the tumor to subsequent ex vivo radiation or chemotherapeutics under conditions wherein the tumor may be in an adapted state. In alternate embodiments, a substantially optimal dose of radiation may be determined by obtaining a tumor tissue sample from a subject, exposing the sample to varying doses of radiation, and monitoring the ability of the tumor tissue sample to undergo an adaptive response at each dose of radiation tested.

In a further aspect, the invention provides a method for identifying small molecule compounds that may be effective chemotherapeutic agents during and after chronic radiation exposure. In certain embodiments, tumor cell lines that are refractory to radiation therapy can be induced to undergo an adaptive response in culture. Such pre-adapted cells may then be subjected to screening for small molecule compounds in search of compounds that are toxic to pre-adapted cells.

Definitions

For convenience, the meaning of certain terms and phrases used in the specification, examples, and appended claims, are provided below.

The term “adaptive response,” as used herein, refers to changes to a cell that render it resistant to the toxic or mutagenic effects of subsequent DNA damage. An adaptive response may be induced by radiation, mitomycin C, hydrogen peroxide, bleomycin, tritiated thymidine, actinomycin D, UV B radiation, and restriction enzymes.

The term “adjuvant therapy,” as used herein, refers to a treatment method used in addition to a primary therapy.

The term “biopsy,” as used herein, refers to the removal of a sample of tissue. A biopsied tissue sample may be examined under a microscope to determine if cancer cells are present and/or subjected to further testing to determine treatment.

The term “DNA damage,” as used herein, refers to any alteration in DNA integrity, such as a base modification, covalent changes to the sugar-phosphate backbone, or a strand break (i.e., single or double-strand break). DNA damage may be caused by exposure to radiation, drugs, toxins, mutagenic chemicals or genetic and/or hereditary disorders.

The term “excision repair,” as used herein, refers to one of three mechanisms that a cell may use to repair damaged DNA. The three modes of excision repair, each of which employs a specialized set of enzymes, include Nucleotide Excision Repair (NER), Base Excision Repair (BER), and Mismatch Repair (MMR). In each mechanism, damaged bases are removed and then replaced with correct bases by localized DNA synthesis.

The terms “gray” or “Gy” refer to a unit of measurement for the amount of radiation energy absorbed by body tissues. A gray is equal to 100 rad and is now the unit of dose. A “centigray”or “cGy” is equal to 1 rad.

The term “inhibition,” as used herein, refers to a delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition, is referred to herein as prevention or eradication.

The terms “internal radiation therapy” or “brachytherapy,” as used herein, refer to a treatment method wherein a source of radioactive material contained in a capsule, pellet, wire, or tube is implanted directly into a tumor or in close proximity to the tumor.

The terms “prevention” and “eradication,” as used herein, refer to no further tumor growth or tumor cell proliferation.

The phrase “therapeutically-effective,” as used herein, is intended to qualify the amount of each agent that will achieve the goal of improvement in neoplastic disease severity and the frequency of incidence over treatment of each agent by itself, while avoiding adverse side effects typically associated with alternative therapies.

The term “tissue sample,” as used herein, refers to any amount of tissue or cells that may be removed during a tissue biopsy, surgery or other standard medical procedure.

The terms “treatment” and “therapy,” as used herein, refer to any process, action, application, or the like, wherein a mammal, including a human being, is subject to medical aid with the object of improving the mammal's condition, directly or indirectly.

The terms “radiation” and “ionizing radiation,” as used herein, refer to energy sources that induce DNA damage, such as gamma-rays, X-rays, UV-irradiation, microwaves, electronic emissions, particulate radiation (e.g., electrons; protons, neutrons, alpha particles, and beta particles), and the like. An irradiating energy source may be carried in waves or a stream of particles or photons. Further, an irradiating energy source has sufficient energy or can produce sufficient energy via nuclear interactions to produce ionization (gain or loss of electrons).

The term “subject,” as used herein, refers to a human or non-human animal.

Certain Methods of the Present Invention

The present invention discloses a method for determining whether a particular tumor undergoes an adaptive response during radiation therapy. Animals treated with chronic low doses of radiation undergo an adaptive response that leads to suppression of homologous recombination to levels that are below those of untreated animals. An increase in certain DNA repair enzymes was also observed following chronic radiation treatment. Tumor cells that are able to undergo such an adaptive response can become resistant to radiation therapy and thus can present additional challenges in the treatment of cancer.

Determining the potential of a tumor or tumor cells to undergo an adaptive response during radiation treatment could be used to determine whether or not radiation therapy or chemotherapeutics given concomitantly with radiation therapy will be an effective method in a patient's treatment for cancer. Methods currently used to determine an effective dose of radiation are empirically derived based on general trends observed among hundreds of cancer patients. However, unlike previous attempts to optimize radiation therapy that have only considered a single exposure of radiation, the methods disclosed herein take into account the duration of radiation treatment and the fact that tumor and/or healthy cells may adapt to chronic radiation exposure. Remarkably, the methods disclosed herein may be used to determine a substantially optimal dose of radiation needed to eradicate a tumor or inhibit tumor growth for individual patients. Optimized dosing regimens of radiation may help to reduce adverse side effects associated with radiation therapy and may be more effective at inhibiting and/or eradicating tumor growth.

The present invention also discloses a method for identifying small molecule compounds (i.e., chemotherapeutic agents) that are effective during and after chronic radiation treatment. The method is based on the insight that cells treated with chronic radiation, which is the normal condition in radiation for cancer, are molecularly distinct from cells that have not undergone chronic radiation treatment. For example, chronic radiation treatment can lead to an adaptive response that makes tumor cells resistant to radiation therapy and to certain chemotherapeutic drugs, particularly chemotherapy drugs that are considered to be “radiomimetic”. On the other hand, alterations in the spectrum of DNA repair enzymes in a tumor cell may also sensitize the radiation-treated tumor cells to certain types of chemotherapeutics. As such, cells treated with chronic radiation may become either resistant or sensitized to different classes of chemotherapeutic drugs, as compared to those cells not treated with radiation.

Remarkably, disclosed herein are a method for determining if a cell has undergone an adaptive response; a method for determining a substantially optimal dose of radiation therapy based on the ability of a cell to undergo an adaptive response; and a method for identifying small molecule compounds that are effective chemotherapeutic agents for use during and after radiation therapy.

Radiation-Induced DNA Damage

Ionizing radiation is frequently administered as a method of treatment for cancer. Radiation therapy can cause DNA damage, such as altered bases, damaged deoxyribose, and single-strand or double-strand breaks (i.e., breakage of phosphodiester bonds) in both healthy and tumor cells. Radiation-induced DNA-damage may result in growth arrest or cell death of the target cells. Growth arrest refers to the inability of a cell to progress to the next phase of the cell cycle. Radiation-induced growth arrest of proliferating cells (i.e., cells actively undergoing cell division or mitosis) and interphase cells (i.e., cells not actively dividing) can lead to cell death by apoptosis or necrosis if the DNA damage is not repaired. The differential ability of healthy cells to repair or tolerate damaged DNA compared to tumor cells is essential for radiation therapy to be an effective treatment for cancer.

DNA damage resulting in altered bases can be repaired by a number of cellular mechanisms including direct chemical reversal of the damage (direct reversal), base excision repair (BER), nucleotide excision repair (NER), and mismatch repair (MMR). Single-strand breaks are repaired primarily by the same enzymes involved in base excision repair. Double-strand DNA breaks can be repaired by two mechanisms: direct joining of DNA by non-homologous end joining (NHEJ); and homologous recombination, which is also known as homology directed repair.

Homology directed repair refers to the process by which double-strand DNA breaks can be repaired using information on sister chromatids, homologous chromosome or the same chromosome. Homology directed repair is also used to reconstitute collapsed replication forks by reinsertion of the broken DNA end. Non-homologous end joining (NHEJ) refers to the joining of free DNA ends. DNA ends may be from the same or different chromosomes and joining of DNA ends originating from non-contiguous sequences causes DNA rearrangements, such as the translocation of pieces of DNA from one chromosome to another.

Ionizing radiation frequently causes double-strand DNA breaks. A double-strand break in a chromosome can jeopardize its physical integrity, which is essential for correct segregation during mitosis and meiosis, as well as for retention of sequence information, which is critical for maintaining accurate encoding of cellular components. While enzymatic cleavage of double-stranded DNA is required for several cellular processes, including recombination during meiosis, V(D)J recombination during immune system development, and mating type switching in S. cerevisiae, double-stranded DNA breaks induced by exposure to radiation, drugs, toxins and mutagenic chemicals can be detrimental to cells and ultimately cellular organisms (e.g., humans). When a cell cannot repair damaged DNA, the cell will induce cell death through apoptosis.

It has been previously observed that cells exposed to chronic radiation therapy may become resistant to further treatment. In the laboratory, cells have been exposed to low doses of radiation as a preconditioning step and then subsequently exposed to a challenging dose of radiation. Studies have also shown that some cells may become resistant to further radiation treatment following a single exposure to radiation. The molecular mechanisms contributing to the radioresistance of pre-exposed cells is unknown.

In the present invention, cells exposed to chronic radiation undergo an adaptive response that results in an increase in DNA repair enzymes and a suppression of homologous recombination to levels below that of untreated cells. Accordingly, during repeated rounds of radiation, healthy cells become resistant to further DNA damage.

An adaptive response may be measured using a variety of cellular end points, including, but not limited to, the suppression of sister chromatid exchanges, mutations, chromosome aberrations, cell survival, apoptosis, micronuclei, and radiation induced thymic lymphomas. Additionally, the induction of “host cell reactivation” of viruses, heat shock protein, homologous recombination, single-strand break repair, double-strand break repair, animal survival and thymine glycol repair may also be a measure of adaptive response.

In certain embodiments, an adaptive response may be measured using cellular markers. An exemplary marker, γ-H2A, has been described in U.S. Pat. No. 6,362,317. H2A histone protein is phosphorylated following a double-strand DNA break and the phosphorylated H2A protein termed, γ-H2A, is involved in the recognition of regions containing double-strand DNA breaks. As disclosed in U.S. Pat. No. 6,362,317, double-strand DNA breaks may be detected using an antibody against γ-H2A.

Other markers that may be used to measure an adaptive response may include key DNA repair enzymes including, but not limited to, Ku70 (Ku autoantigen protein p70), Ape1 (AP endonuclease 1), and Pol β (polymerase β). Additionally, the expression levels of ERCC1 (excision repair cross-complementing rodent repair deficiency, complementation group 1) and XPF (also known as ERCC4, excision repair cross-complementing rodent repair deficiency, complementation group 4) may be upregulated during an adaptive response, and thus, may be measured as a marker of adaptation. In certain embodiments, the expression levels of markers used to measure an adaptive response including, but not limited to, Ku70, Ape1, Pol β, ERCC1 and XPF may be assessed by Western blotting. In an alternate embodiment, the expression levels of adaptation markers, such as those listed above, may be assessed by immunohistochemistry to measure an adaptive response.

Additional assays compatible with immunohistochemical staining of tissue may also be used to detect an adaptive response. For example, antibody-based assays may be used to assess the response of tumor and/or healthy cells to radiation-induced toxicity in tissue sections containing both cell types. One exemplary antibody that may be used is an antibody against Mre11. In healthy cells, Mre11 is diffuse in the cells, but in damaged cells, Mre11 is recruited at high concentrations into foci that form at double-strand DNA breaks. At double-strand breaks, Mre11 forms a complex with Rad50 and Nbs1 and these foci can be readily detected by antibody staining. While it has been proposed that persistant Mre11 foci reflect poorly repaired double strand breaks (e.g., foci remain present 8 hours after irradiation), it has been shown that adapted cells have an accelerated clearance of double strand DNA breaks. Thus, the levels of Mre11 foci may be a measurable endpoint to determine the response of tumor and/or healthy cells to radiation.

Another exemplary antibody that may be used in an immunohistochemistical assay is an antibody against hPso4. Like the markers discussed above, hPso4 protien levels are increased (e.g., hPso4 is increased 15 to 30 fold) following exposure to DNA damaging agents that cause double-strand DNA breaks.

One of ordinary skill in the art will appreciate that additional markers may be identified that are either up-regulated or down-regulated in adapted cells as compared to unexposed cells; therefore, these markers may also be used to assess adaptation.

In a further embodiment, an adaptive response may be monitored by comparing the transcriptome of an adapted cell to the transcriptome of an unexposed cell. Not wishing to be bound by theory, it is likely that the spectrum of RNA expression will differ between chronically irradiated cells and untreated cells.

Other markers of adaptation could include changes in the following end points: biochemical assays for DNA damage (e.g., assays for double-stranded breaks or other types of DNA lesion detection), movement of repair proteins in response to damage (such as formation of DNA repair foci), changes in membrane permeability (both intracellular and cellular membranes), markers of apoptosis (e.g., caspase activity, nuclear morphology, etc.), and markers of necrosis.

In a still further embodiment, in situ detection of strand breaks may also be used to assess an adaptive response following radiation exposure. For example, variations on the TUNEL assay, which labels the free 3′—OH that is present on broken ends of the duplex DNA (a 3′OH is present at DNA ends, whether they are single or double stranded, blunt, overhanging or recessed), may be used. All variations of the TUNEL assay exploit the terminal deoxynucleotidyl transferase (TdT) enzyme, which is a template-independent polymerase that is able to add deoxyribonucleotides to DNA ends (e.g., strand breaks). In an assay to detect double-strand breaks, ends may be labeled by incorporating biotinylated deoxyuridine at 3′—OH ends, followed by using streptavidin-linked fluorescence molecules to bind the ends; alternatively, ends may be labeled by adding fluorescently tagged dU (deoxyuridine).

Additional assays to detect an adaptive response include toxicity assays. Exemplary toxicity assays include colony-forming assays, DNA synthesis assays, and vital dye uptake assays. In a colony-forming assay, cultured cells are dispersed and counts of the number of cells able to form colonies are made to determine the response of the cells to radiation. The ability to form colonies is a measure of the effect of radiation, since effective treatment of cancer requires that treatment conditions eradicate the ability of tumor cells to proliferate. In DNA synthesis assays, the measure of toxicity is the inability of cells to replicate their DNA during S phase. This may be measured by adding bromodeoxyuridine (BrdU) to cultured cells or explants and then labeling with anti-BrdU antibodies. In vital dye uptake assays, the premise is that living cells can actively transport dye into intracellular membrane bound compartments in which enzymatic activity can be detected. For example, a dye may be used that is partially transported to the mitochondria of living cells. Once in the mitochondria the dye is cleaved to render it optically detectable (i.e., the dye is transformed from colorless to colored). An example of such an assay is MTT (a tetrazolium-based colorimetric assay).

Additional assays to detect an adaptive response include apoptosis assays. Exemplary apoptosis assays include labeling of annexin V, which is an antigen present on apoptotic cells, and caspase cleavage assays. Assays based on caspase 3, 6, and/or 8 may provide sensitive approaches for detecting the activity of enzymes that are activated by apoptosis.

In a still further embodiment, inflammation, initiation of an inflammatory response or release of inflammatory chemicals may increase the levels of homologous recombination in normal cells (e.g., normal mammalian and microbial cells). In certain embodiments, inflammatory chemicals may act as DNA damaging agents. In mammalian cells, DNA damaging agents released during inflammation may be among the most “recombinogenic” (i.e., leading to some degree of recombination) chemical exposures endured by individuals. As such, we have identified DNA repair proteins that prevent inflammatory chemicals in microbial cells. Further, we have identified a chemical pathway by which inflammation induces homologous recombination and modulates the risk of inflammation-induce sequence arrangements in microbial and mammalian cells.

Similarly, cancer chemotherapeutics are DNA-damaging agents and can promote secondary cancers long after treatments. In particular, several common chemotherapeutic agents cause cells to undergo high levels of homologous recombination and can significantly increase the risk of tumorigenic sequence rearrangements. We have revealed that a cancer chemotherapeutic can change the state of a cell causing it to have an increased risk of homologous recombination weeks after a single acute exposure. Thus, cells may communicate with each other inducing homologous recombination from one cell to the next in a sequential fashion. This mechanism may account for the genetic instability that results from an acute (e.g., single) exposure to a DNA damaging agent, sometimes leading to the development of cancer long after the acute exposure.

In certain embodiments, recombination assays that reveal the types of sequence rearrangements induced by different exposures to chemotherapeutic agents may be used. In particular, interstrand crosslinks preferentially induce non-conservative recombination events (e.g., pathways that are associated with crossovers and long-tract-break-induced-replication). This outcome is in contrast to most spontaneous recombination events, which are highly conservative (short tract gene conversions without associated crossover). Chemical agents may cause persistent hyper-recombination, leading to an increased rate of recombination of more than 2, 5, 10, 20, 40, 80 or more cell doublings after exposure. The progeny of damage-exposed cells may also induce a similarly high rate of recombination among neighboring cells, which may in turn induce recombination to a similar extent in the neighboring cells. Thus, damage may induce recombination in a DNA lesion-independent fashion.

Determining an Adaptive Response during Chronic Radiation Therapy

The present invention discloses a method for discerning an adaptive response of a tumor during chronic radiation therapy. Determining the potential of a tumor or tumor cells to undergo an adaptive response during chronic radiation treatment could be predictive of whether or not radiation therapy will be an effective treatment for a subject. In certain embodiments, a method for determining an adaptive response of a tumor during chronic radiation therapy may comprise administering radiation treatment to a subject prior to surgery; removing a tumor or part of the tumor from the subject during surgery; and monitoring a sample of the tumor tissue for an adaptive response. Optionally, a healthy tissue sample may also be removed during surgery and monitored for an adaptive response. The ability of healthy tissue to undergo an adaptive response may be compared to the ability of the tumor tissue to undergo an adaptive response.

In an exemplary embodiment, a tumor tissue sample will be obtained during surgery. Prior to surgery, a subject may be exposed to one or more individual doses of radiation ranging from about 0.25 to about 5.0 Gy and the total dose of radiation may range from about 0.25 to about 100 Gy. In certain embodiments, a subject may be exposed to doses of radiation ranging from about 0.25 to 0.5 Gy, about 0.5 to 1.0 Gy, about 1.0 to 1.5 Gy, about 1.5 to 2.0 Gy, about 2.0 to 2.5 Gy, about 2.5 to 3.0 Gy, about 3.0 to 3.5 Gy, about 3.5 to 4.0 Gy, about 4.0 to 4.5 Gy, and about 4.5 to 5.0 Gy. An exemplary dose of radiation to monitor an adaptive response is from about 1.0 to about 3.0 Gy. A subject may be exposed to a single dose or multiple doses of radiation prior to surgery. In an exemplary embodiment, the subject may be exposed to four or five doses of radiation prior to surgery.

In a further embodiment, any source of ionizing radiation well known in the art may be used for radiation therapy (see below). In an exemplary embodiment, x-rays may be the type of radiation treatment administered to the subject. The length of exposure time required to reach the dose ranges listed above will depend on the radiation source. Exposure times may range from about one minute to about five minutes.

Following exposure to radiation, a tumor tissue sample may be removed from a subject during surgery to remove a tumor mass. Optionally, a healthy tissue sample may also be removed from a subject during surgery to remove a tumor mass. Tumor and/or healthy tissue may then be used as single tissue samples or divided into multiple tissue samples. To determine if an adaptive response occurred during the initial exposure to radiation before surgery, the expression levels of genes known to upregulated during an adaptive response may be measured. Alternatively, other indications of DNA damage or responses to DNA damage may be measured. Further, to determine if an adaptive response will occur in subsequent rounds of radiation after surgery, tissue samples may be treated with subsequent doses of radiation ex vivo to monitor the ability of the tissue samples to undergo an adaptive response.

In an exemplary embodiment, the expression levels of genes known to be upregulated during an adaptive response may be measured following surgery to remove a tumor pre-exposed to radiation treatment. An adaptive response in the tissue samples may be determined by measuring γ-H2A as described in U.S. Pat. No. 6,362,317 (incorporated by reference). Briefly, γ-H2A may be detected using a variety of methods known in the art. Such methods may use an anti-γ-H2A antibody to detect γ-H2A in chromatin or reconstituted chromatin, protein extracts, or whole cells. Binding of anti-γ-H2A antibody to a sample may be quantified. Detection of γ-H2A indicates the presence of a double-strand DNA break. Further, tissue samples that show a quantifiable amount of anti-γ-H2A antibody binding would indicate an increase in DNA damage via double-strand DNA breaks and sensitivity to radiation therapy. Tissue samples that do not show a quantifiable amount of anti-γ-H2A antibody binding would indicate minimal DNA damage and a resistance to radiation therapy.

Additional markers may also be measured to assess whether or not the tissue sample adapted to radiation. Such markers, as described above, may include, but are not limited to, Ku70, Ape1, Pol β, ERCC1 and XPF. Increased expression of each of these markers may be monitored by Western blotting and/or immunohistochemistry. Other markers may also be used and may include other assays for detecting double-strand DNA breaks as described above.

In another exemplary embodiment, tumor and/or healthy tissues obtained during surgery following a course of radiation treatment may be divided into multiple samples. Each tissue sample may then be treated ex vivo with subsequent doses of radiation to determine if the tissue sample undergoes an adaptive response with further radiation treatment. A combinatorial approach may be used to test a range of radiation doses, the number of doses to be administered, and the length of each radiation dose on parallel tissue samples. In certain embodiments, the tissue samples may be exposed to doses ranging from about 0.25 to about 5.0 Gy. Doses of radiation may range from about 0.25 to 0.5 Gy, about 0.5 to 1.0 Gy, about 1.0 to 1.5 Gy, about 1.5 to 2.0 Gy, about 2.0 to 2.5 Gy, about 2.5 to 3.0 Gy, about 3.0 to 3.5 Gy, about 3.5 to 4.0 Gy, about 4.0 to 4.5 Gy, and about 4.5 to 5.0 Gy. In further embodiments, the tissue samples may be exposed to varying doses of radiation ranging from a single dose to five doses of radiation. In a still further embodiment, the length of each dose may also be varied, ranging from one minute to 5 minutes for each dose. Further, the source of radiation may also vary. Different radiation sources may be introduced into the assay, to determine if an adaptive response occurs preferentially with a particular source of radiation.

An adaptive response may be measured following subsequent treatment with radiation by measuring amount of anti-γ-H2A antibody that can be bound by the samples. For example, tissue samples that have undergone an adaptive response may show a reduced number of γ-H2A foci following subsequent irradiation. Alternatively, the expression levels of adaptation markers, such as Ku70, Ape1, Pol β, ERCC1 and/or XPF may be measured to assess whether or not an adaptive response has occurred during subsequent irradiation. Additional assays to detect double-strand DNA breaks as described above may also be employed.

Alternatively, an adaptive response in the tissue maintained under ex vivo conditions may be monitored by measuring cell survival. For example, a survival curve, which plots the number of surviving cells versus the total acute dose of radiation, may be generated. Alternatively, cell survival may be measured using a marker of toxicity (e.g., cytoxicity curves may be generated to plot toxicity versus the total acute dose of radiation). Tumor tissue or neoplastic cells are expected to show a decrease in survival (or increased toxicity) when they are first irradiated. After multiple doses, the cells may become resistant to toxicity if they have undergone an adaptive response. Tumor tissue that does not show a decrease in cell survival following pre-exposure would be indicative of cells that are undergoing an adaptive response and may therefore be resistant to further radiation therapy.

Healthy tissue and tumor tissue may respond similarly in their ability to mount an adaptive response. Quantifying the ability of healthy and tumor tissues may be indicative of how well a subject will ultimately respond to radiation therapy. In an exemplary embodiment, healthy tissues would be able to mount an adaptive response, whereas tumor tissues would remain susceptible to radiation therapy.

In an alternate embodiment, a method for determining an adaptive response of a tumor during chronic radiation therapy may comprise obtaining a tumor tissue sample from a subject; exposing the tumor tissue sample to radiation ex vivo; and monitoring the tumor tissue for an adaptive response. In a further embodiment, a healthy tissue sample may also be obtained from a subject. The ability of healthy tissue to undergo an adaptive response may be compared to the ability of the tumor tissue to undergo an adaptive response.

In an exemplary embodiment, a tumor tissue sample may be obtained during a biopsy procedure. Optionally, a healthy tissue sample may also be obtained during a biopsy procedure. Biopsied tissue may then be irradiated under ex vivo conditions. Biopsied tissue may be exposed to doses of radiation ranging from about 0.25 to about 5 Gy. In certain embodiments, the dose of radiation may range from about 0.25 to 0.5 Gy, about 0.5 to 1.0 Gy, about 1.0 to 1.5 Gy, about 1.5 to 2.0 Gy, about 2.0 to 2.5 Gy, about 2.5 to 3.0 Gy, about 3.0 to 3.5 Gy, about 3.5 to 4.0 Gy, about 4.0 to 4.5 Gy, and about 4.5 to 5.0 Gy. An exemplary dose of radiation to monitor an adaptive response is from about 1.0 to 3.0 Gy. Biopsied tissue may be exposed to a single dose of radiation or to multiple doses of radiation. In an exemplary embodiment, biopsied tissue may be exposed to four or five doses of radiation.

In a further embodiment, any source of ionizing radiation may be used irradiate the biopsied tissue. In an exemplary embodiment, x-rays may be used to irradiate the biopsied tissue. The length of exposure time required to reach the dose ranges listed above will depend on the radiation source. Exposure times may range from about one minute to about five minutes.

In certain embodiments, a combinatorial approach may be used to test a range of radiation dosages, the number of doses to be administered, and the length of each radiation dose on parallel tissue samples.

Under ex vivo conditions, an adaptive response in the biopsied tissue may be monitored by measuring the amount of anti-γ-H2A antibody that can be bound by the samples. As described above any number of methods well known in the art may be employed to detect anti-γ-H2A antibody binding to γ-H2A protein. Biopsied tissue that shows an increase in anti-γ-H2A antibody binding would indicate increased DNA damage via double-strand DNA breaks and sensitivity to radiation therapy. Biopsied tissue that does not show an increase in anti-γ-H2A antibody binding would indicate minimal DNA damage and resistance to radiation therapy. Further, pre-irradiation with one or more doses may reduce the number of γ-H2A foci in the tissue following a subsequent irradiation.

Alternatively, an adaptive response in the biopsied tissue maintained under ex vivo conditions may be monitored by measuring cell survival. For example, a survival curve, which plots the number of surviving cells versus the total acute dose of radiation, may be generated. Alternatively, cell survival may be measured using a marker of toxicity (e.g., cytoxicity curves may be generated to plot toxicity versus the total acute dose of radiation). Biopsied tissue or neoplastic cells are expected to show a decrease in survival (or increased toxicity) when they are first irradiated. After multiple doses, the cells may become resistant to toxicity if they have undergone an adaptive response. Biopsied tissue that does not show a decrease in cell survival would be indicative of cells that are undergoing an adaptive response and may therefore be resistant to radiation therapy.

Healthy and tumor tissue may respond similarly to radiation therapy in their ability to mount an adaptive response. A differential adaptive response between healthy and tumor tissue may be indicative of the effectiveness of radiation therapy. In an exemplary embodiment, healthy tissues would be able to mount an adaptive response, whereas tumor tissues would remain susceptible to radiation therapy.

The present invention also discloses a method for determining a substantially optimal dose of radiation needed to eradicate a tumor or inhibit tumor growth. In certain embodiments, a method for determining a substantially optimal dose of radiation therapy comprises administering radiation therapy to a subject prior to removing a tumor by surgery; removing the tumor by surgery; and exposing the tumor tissue to varying doses of radiation ex vivo to determine if an adaptive response may occur in the tumor tissue. Optionally, a healthy tissue sample may also be obtained and exposed to varying doses of radiation to determine if an adaptive response occurs in the healthy tissue cells.

Prior to surgery a subject may be exposed to one or more individual doses of radiation ranging from about 0.25 to about 5.0 Gy and the total dose of radiation may range from about 0.25 to about 100 Gy. In certain embodiments, the dose of radiation may range from about 0.25 to 0.5 Gy, about 0.5 to 1.0 Gy, about 1.0 to 1.5 Gy, about 1.5 to 2.0 Gy, about 2.0 to 2.5 Gy, about 2.5 to 3.0 Gy, about 3.0 to 3.5 Gy, about 3.5 to 4.0 Gy, about 4.0 to 4.5 Gy, and about 4.5 to 5.0 Gy. In an exemplary embodiment, the dose of radiation is from about 1.0 to about 3.0 Gy. In a further embodiment, the radiation therapy administered to a subject prior to surgery comprises between one to five doses of radiation therapy. In an exemplary embodiment, four or five doses of radiation are administered prior to surgery. Any source of radiation may be used and the length of exposure will depend on the source of radiation used. In an exemplary embodiment, x-rays are used as the source of radiation prior to surgery.

In an alternate embodiment, a method for determining a substantially optimal dose of radiation therapy comprises obtaining a tumor tissue sample from a subject; exposing the tumor tissue sample to varying dosages of radiation ex vivo; and determining if an adaptive response occurs in the tumor tissue sample. Optionally, a healthy tissue sample may also be obtained and exposed to varying doses of radiation ex vivo to determine if an adaptive response occurs in the healthy tissue cells.

Tissue samples obtained during surgery following radiation treatment or during biopsy without prior exposure to radiation may be divided to generate subpopulations of cells that may be tested with varying doses of radiation. Doses of radiation may range from about 0.25 to about 5 Gy. In certain embodiment, radiation doses may range from about 0.25 to 0.5 Gy, about 0.5 to 1.0 Gy, about 1.0 to 1.5 Gy, about 1.5 to 2.0 Gy, about 2.0 to 2.5 Gy, about 2.5 to 3.0 Gy, about 3.0 to 3.5 Gy, about 3.5 to 4.0 Gy, about 4.0 to 4.5 Gy, and about 4.5 to 5.0 Gy. In an exemplary embodiment, multiple doses may be tested ranging from low doses of radiation to high doses of radiation. Each subpopulation of cells may be exposed to single dose or multiple doses of radiation. In an exemplary embodiment, each subpopulation of cells may be exposed to four or five doses of radiation.

In a further embodiment, any source of ionizing radiation may be used to irradiate the subpopulation of tumor and/or healthy tissue cells. In an exemplary embodiment, x-rays are used to irradiate the subpopulations of tumor and/or healthy tissue cells. The length of exposure time will depend on the radiation source. Exposure times may range from about one minute to about five minutes.

In certain embodiments, a combinatorial approach on parallel samples may be used to test a range of radiation dosages, the number of doses to be administered, and the length of each radiation dose.

Following exposure to radiation, an adaptive response may be monitored by measuring the amount of anti-γ-H2A antibody that can be bound by the cell samples, as described above, or by measuring the expression of Ku70, Ape1, Pol β, ERCC1 and/or XPF. Alternatively, an adaptive response may be monitored by measuring cell survival, also as described above. Further, an assay described above may be used to assess an adaptive response.

In an exemplary embodiment, tumor tissues remain sensitive to radiation therapy after repeated dosing. After a tumor has been determined to be radiosensitive (i.e., the tumor does not undergo an adaptive response), a substantially optimal dose of radiation may be determined based on the conditions tested on the tumor and/or healthy tissue sample. Radiation therapy may then be administered based on the optimized dosing regimen. In an exemplary embodiment, the optimized dose would be a minimal effective dose of radiation that produces an increase in cell damage or death in tumor cells while minimizing adverse side effects in normal tissue.

An optimized dose of radiation therapy may be given to a subject as a daily dose. Optimized daily doses of radiation therapy may be from about 0.25 to 0.5 Gy, about 0.5 to 1.0 Gy, about 1.0 to 1.5 Gy, about 1.5 to 2.0 Gy, about 2.0 to 2.5 Gy, and about 2.5 to 3.0 Gy. An exemplary daily dose may be from about 2.0 to 3.0 Gy. A higher dose of radiation may be administered if a tumor is resistant to lower doses of radiation. High doses of radiation may reach 4 Gy. Further, the total dose of radiation administered over the course of treatment may range from about 50 to 200 Gy. In an exemplary embodiment, the total dose of radiation administered over the course of treatment ranges from about 50 to 80 Gy. In certain embodiments, a dose of radiation may be given over a time interval of 1, 2, 3, 4, or 5 minutes, wherein the amount of time is dependent on the dose rate of the radiation source.

In certain embodiments, a daily dose of optimized radiation may be administered 4 or 5 days a week, for approximately 4 to 8 weeks. In an alternate embodiment, a daily dose of optimized radiation may be administered daily seven days a week, for approximately 4 to 8 weeks. In certain embodiments, a daily dose of radiation may be given a single dose. Alternately, a daily dose of radiation may given as a plurality of doses. In a further embodiment, the optimized dose of radiation may be a higher dose of radiation than can be tolerated by the patient on a daily base. As such, high doses of radiation may be administered to a patient, but in less frequent dosing regimen.

The types of radiation that may be used in cancer treatment are well known in the art and include electron beams, high-energy photons from a linear accelerator or from radioactive sources such as cobalt or cesium, protons, and neutrons. An exemplary ionizing radiation is an x-ray radiation.

Methods to administer radiation are well known in the art. Exemplary methods include, but are not limited to, external beam radiation, internal beam radiation, and radiopharmaceuticals. In external beam radiation, a linear accelerator is used to deliver high-energy x-rays to the area of the body affected by cancer. Since the source of radiation originates outside of the body, external beam radiation can be used to treat large areas of the body with a uniform dose of radiation. Internal radiation therapy, also known as brachytherapy, involves delivery of a high dose of radiation to a specific site in the body. The two main types of internal radiation therapy include interstitial radiation, wherein a source of radiation is placed in the effected tissue, and intracavity radiation, wherein the source of radiation is placed in an internal body cavity a short distance from the affected area. Radioactive material may also be delivered to tumor cells by attachment to tumor-specific antibodies. The radioactive material used in internal radiation therapy is typically contained in a small capsule, pellet, wire, tube, or implant. In contrast, radiopharmaceuticals are unsealed sources of radiation that may be given orally, intravenously or directly into a body cavity.

Radiation therapy may also include sterotactic surgery or sterotactic radiation therapy, wherein a precise amount of radiation can be delivered to a small tumor area using a linear accelerator or gamma knife and three dimensional conformal radiation therapy (3DCRT), which is a computer assisted therapy to map the location of the tumor prior to radiation treatment.

Optimized radiation therapy may be used as adjuvant therapy to surgery. In certain embodiments, radiation therapy will be administered at the time of surgery (i.e., intraoperative therapy) directly to the effected area. In other embodiments, radiation may be administered prior to surgery to reduce the size or shrink a tumor before it can be surgically removed. In further embodiments, radiation may be administered following surgery to kill any cancerous cells that may remain at the site of the tumor and/or prevent further tumor growth or metastasis. In still further embodiments, radiation may be administered in any combination of pre-surgery therapy, intraoperative therapy, or post-surgery therapy.

An optimized dosing regimen of radiation therapy may be combined temporally with chemotherapy to improve the outcome of treatment. There are various terms to describe the temporal relationship of administering radiation therapy and chemotherapy. The administration of combined radiation and chemotherapy is often called radiochemotherapy. The following examples are exemplary treatment regimens, which are generally known by those skilled in the art, and are provided for illustration only and are not intended to limit the use of other combinations. “Sequential” radiation therapy and chemotherapy refers to the administration of chemotherapy and radiation therapy separately in time in order to allow the separate administration of either chemotherapy or radiation therapy. “Concomitant” radiation therapy and chemotherapy refers to the administration of chemotherapy and radiation therapy on the same day. Finally, “alternating” radiation therapy and chemotherapy refers to the administration of radiation therapy on the days in which chemotherapy would not have been administered if it was given alone.

In certain embodiments, an optimized dosing regimen of radiation therapy may be combined with chemotherapy and surgery. In certain embodiments, following adaptation by exposure to radiation, chemotherapeutics may be identified that are most toxic to the tumor under the adapted conditions. This identification can be based upon the gene expression pattern of the tumor following pre-exposure to radiation, or upon ex vivo testing of the sensitivity of the tumor cells to chemotherapeutic agents following pre-exposure to radiation.

In an exemplary embodiment, tumor and/or healthy tissues obtained during surgery following a course of radiation treatment may also be treated ex vivo with chemotherapeutic drugs. In certain embodiments, tumor and/or healthy tissue samples may be divided to generate subpopulations of cells that may be tested with various chemotherapeutic drugs. A combinatorial approach may be used to test a range of chemotherapeutic drugs at varying dosages. Sensitivity or resistance of the radiation treated cells to a particular chemotherapeutic drug may be monitored by measuring cell survival or toxicity as described above. An optimized dose of radiation may then be combined with an effective chemotherapeutic agent to increase cell damage or death in tumor cells.

Chemotherapeutic drugs that may be tested as described above and/or administered temporally with an optimized dose of radiation therapy may include, but are not limited to, alkylating agents, antiestrogens, aclarubicin, actinomycin D, aldesleukin, alemtuzumab, alitretinoin, allopurinol, altretamine, amifostine, anastrozole, asparaginase, bexarotene, bisantrene, bleomycin, busulfan, BCNU (carmustine), calusterone, capecitabine, carboplatin, celecoxib, chlorambucil, cisplatin, cladribine, cyclophosphamide, cyclooxygenase-2 inhibitor, cytarabine, CCNU (lomustine), dacarbazine, daunorubine, daunomycin, denileukin diftitox, dexrazoxane, diaziquone, docetaxel, doxorubicin, epirubicin, epoetin alfa, esorubicin. estramustine, etoposide (VP-16), exemestane, Filgrastim, floxuridine, fludarabine, 5-fluorouracil, fulvestrant, galactitol, gemcitabine, gemtuzumab, goserelin acetate, hydroxyurea, ibritumomab tiuxetan, idarubicin, ifosfamide, imatinib mesylate, interferon alpha, interferon gamma, iriniotecan, iroplatin, letrozole, leucovorin, levamisole, lonidamine, megrestrol acetate, melphalan, mercaptopurine, mesna, methotrexate, methoxsalen, mitomycin C, mitotane, mitoxantrone, mitoguazone, nandrolone phenpropionate, Nofetumomab, nitrogen mustard, oprelvekin, oxaliplatin, paclitaxel, pamidronate, pegademase, pegaspargase, pegfilgrastim, pentostatin, pipobroman, plicamycin, porfimer sodium, procarbazine, progestins, prednimustine, PCNU, quinacrine, rasburicase, rituximab, sargramostim, streptozocin, talc, tamoxifen, temozolomide, teniposide (VM-26), testolactone, thioguanine, thiotepa, topotecan, toremifene, tositumomab, trastuzumab, tertinoin, uracil mustard, valrubicin, vinblastine, vincristine, vindesine, vinorelbine, and zoledronate.

Tumors that can be treated with the present invention include, but are not limited to, tumors of the breast, colon, lung, liver, lymph node, kidney, pancreas, prostate, ovary, endometrium, spleen, small intestine, stomach, skin, testes, head and neck, esophagus, brain (glioblastomas, medulloblastoma, astrocytoma, oligodendroglioma, ependymomas), blood cells, bone marrow, blood cells, blood or other tissue. The tumor may be distinguished as metastatic or non-metastatic. Various embodiments include, but are not limited to, tumor cells of the breast, colon, lung, liver lymph node, kidney, pancreas, prostate, ovary, endometrium, spleen, small intestine, stomach, skin, testes, head and neck, esophagus, brain, bone marrow or blood cells. Other embodiments include fluid samples, such as peripheral blood, lymph fluid, ascites, serous fluid, pleural effusion, sputum, cerebrospinal fluid, lacrimal fluid, stool or urine.

In an exemplary embodiment, the method for treating a tumor comprises treating a subject with radiation therapy wherein the tumor is selected from breast cancer, colon cancer, lung cancer, gastrointestinal cancer, ovarian cancer, prostate cancer, head and neck cancer, liver cancer, and cervical cancer.

The methods and combinations of the present invention may also be used for the treatment of neoplasia disorders selected from the group consisting of acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangiocarcinoma, chondrosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastoma; renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiatied carcinoma, uveal melanoma, verrucous carcinoma, vipoma, well differentiated carcinoma, and Wilm's tumor.

Identification of Chemotherapeutic Drugs Using Pre-Adapted Target Cells

As cells undergo an adaptive response following chronic irradiation, the expression of certain DNA repair enzymes is increased and homologous recombination is suppressed. The increased expression of DNA repair enzymes, however, is not always protective for a cell and can actually increase the sensitivity of the cell to certain DNA damaging agents. Further, the toxicity of some DNA damaging agents may be enhanced when a cell attempts to repair the damaged DNA lesion. For example, the toxicity of ET743 largely depends on a cell's ability to perform nucleotide excision repair (NER). Cells lacking NER are resistant, while cells proficient in NER are sensitive. Induction of NER by ionizing radiation or UV light may sensitize a tumor or tumor cells to ET743. Thus, ET743 may be more effective in patients if administered after starting a course of radiotherapy, than if ET743 is administered as an independent treatment.

Thus, the present invention provides a method for identifying chemotherapeutic drugs that are effective during and after radiation therapy that uses cells pre-adapted to chronic radiation exposure to screen for small molecule compounds that induce DNA damage and/or cell death in the pre-adapted cells. Not wishing to be bound by theory, it is likely that under conditions of chronic irradiation, compounds that are marginally toxic to an unadapted tumor cell may become extremely toxic to the adapted tumor cell.

In certain embodiments, tumor cells that are refractory to radiation treatment (i.e., undergo an adaptive response) may be adapted by exposure to ionizing radiation prior to screening. Adaptation can also be induced by exposure to several other agents, including reactive oxygen generating drugs, agents that induce double strand breaks, and crosslinking agents. Examples of tumor cells that are refractory to radiation treatment include, but are not limited to, GL-13 cells (a Glioblastoma multiforme derived cell line), JW-1T cells (a Glioblastoma multiforme derived cell line), and SCC-61 cells (derived from head and neck squamous cell carcinoma).

In further embodiments, any number of cultured cells may be adapted and screened for small molecule compounds that induce DNA damage and cell death in the pre-adapted cells. The type of cultured cells that may be used in the screen described herein is not critical to the screening method of the present invention; however, cultured cells that are amendable to screening will undergo an adaptive response as described below.

The following exemplary assay may be used to determine if a particular cultured cell undergoes an adaptive response. For example, cells grown in culture may be exposed to a dose of radiation ranging from about 0.25 to about 5.0 Gy. An initial test dose may for instance be about 2.0 Gy. Following the initial dose of radiation, a subset of the cells may be assessed for their ability to respond to the radiation by measuring cell survival or cell toxicity (see below). Exposed cells should show a decrease in cell survival compared to the unexposed cells indicating that they are sensitive to radiation treatment. Remaining cells are subsequently exposed to repeated doses of radiation and assayed for an adaptive response after each dose of radiation. In our exemplary assay, culture cells are exposed to five doses of radiation. Cells undergoing an adaptive response will show diminished cell killing with each round of radiation exposure and may show no difference in cell killing in the final round of radiation exposure. Those of ordinary skill of the art will appreciate that the described assay may be modified to test varying doses of radiation.

Sources of ionizing radiation may include, but are not limited to, x-rays, gamma-rays, ultraviolet irradiation, and microwave irradiation. As increasing doses of ionizing radiation may differentially sensitize cells to small molecule compounds, assays may be performed at radiation doses ranging from about 0.25 to 0.5 Gy, about 0.5 to 1.0 Gy, about 1.0 to 1.5 Gy, about 1.5 to 2.0 Gy, about 2.0 to 2.5 Gy, about 2.5 to 3.0 Gy, about 3.0 to 3.5 Gy, about 3.5 to 4.0 Gy, about 4.0 to 4.5 Gy, and about 4.5 to 5.0 Gy. Screening assays may also be modified to test radiation doses higher than 5.0 Gy. In an exemplary embodiment, doses of radiation to pre-adapt cells may be similar to clinically relevant doses of radiation. Exemplary doses of radiation may range from about 1.0 Gy to about 3.0 Gy.

For pre-adaptation to radiation, cells will be treated with one or more exposures of radiation at the elected radiation dose. In an exemplary embodiment, cells will be treated 4 or 5 times with ionizing radiation. The length of exposure may range from about 30 seconds to about 5 minutes and exposure time will be maintained for each repeated dose. Exposures greater than 5 minutes may also be used as long as the cells remain amendable to screening. The exposure time will depend upon the source of radiation.

In an alternate embodiment, adaptation can also be induced by exposure to several other agents including, but not limited to, reactive oxygen generating drugs, agents that induce double-strand DNA breaks, and cross-linking agents. Reactive oxygen generating drugs that may be used to induce an adaptive response include, but are not limited to, all forms of radiation, redox cycling drugs (including mitomycin C and other polycyclic aromatic hydrocarbons), hydrogen peroxide, benzoil peroxide, and phenyloin. Agents that either directly or indirectly cause formation of double-strand DNA breaks that may be used to induce an adaptive response include, but are not limited to, BCNU (carmustine), CCNU (loumustine), cisplatin, oxaloplatin, melphalan, nitrogen mustards, bleomycin, teniposide, and other agents that covalently modify DNA. Cross-linking agents that may be used to induce an adaptive response include, but are not limited to, BCNU (carmustine), CCNU (lomustine), cisplatin, oxaloplatin, melphalan, and nitrogen mustards.

Pre-adaptation may be monitored by using a survival curve that plots cell survival versus total acute dosage of radiation under conditions where cells receive daily doses or chronic irradiation. If the cells adapt, then the extent of toxicity induced by the first exposure will be significantly greater than the extent of toxicity induced by subsequent exposures. Pre-adapted cells may be maintained using cell culture conditions appropriate for non-adapted cells. Basic cell culture conditions are well known in the art.

Pre-adapted cells treated with small molecule compounds may be monitored for increased DNA damage and/or cell death using a variety of methods. Such methods may include monitoring cell survival or cell count, induction of apoptosis, or a DNA-damage induced cell marker (i.e., expression of γ-H2A protein). Additional markers that are indicative of an adaptive response may include, but are not limited to, Ku70, Ape1, Pol β, ERCC1 and XPF.

Small molecule compounds that that induce DNA damage and/or cell death in the pre-adapted cells may be identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N. H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmnaMar, U.S.A. (Cambridge, Mass.). In addition, natural and synthetically-produced libraries can be generated, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their chemotherapeutic activity should be employed whenever possible.

When a crude extract is found to induce DNA damage and/or cell death in pre-adapted cells, further fractionation of the positive lead extract is necessary to isolate chemical constituents responsible for the observed effect. Thus, the goal of the extraction, fractionation, and purification process is the careful characterization and identification of a chemical entity within the crude extract capable of inducing DNA damage and/or cell death. Methods of fractionation and purification of such heterogeneous extracts are known in the art.

Known chemotherapeutic agents may also be tested. In such instances, certain known chemotherapeutic agents may be identified to show an enhanced effect after administration of a given dose of radiation. Ideally an enhanced effect would be synergistic. Chemotherapeutic agents that may be tested include, but are not limited to, alkylating agents, antiestrogens, aclarubicin, actinomycin D, aldesleukin, alemtuzumab, alitretinoin, allopurinol, altretamine, amifostine, anastrozole, asparaginase, bexarotene, bisantrene, bleomycin, busulfan, BCNU (carmustine), calusterone, capecitabine, carboplatin, celecoxib, chlorambucil, cisplatin, cladribine, cyclophosphamide, cyclooxygenase-2 inhibitor, cytarabine, CCNU (lomustine), dacarbazine, daunorubine, daunomycin, denileukin diftitox, dexrazoxane, diaziquone, docetaxel, doxorubicin, epirubicin, epoetin alfa, esorubicin, estramustine, etoposide (VP-16), exemestane, Filgrastim, floxuridine, fludarabine, 5-fluorouracil, fulvestrant, galactitol, gemcitabine, gemtuzumab, goserelin acetate, hydroxyurea, ibritumomab tiuxetan, idarubicin, ifosfamide, imatinib mesylate, interferon alpha, interferon gamma, iriniotecan, iroplatin, letrozole, leucovorin, levamisole, lonidamine, megrestrol acetate, melphalan, mercaptopurine, mesna, methotrexate, methoxsalen, mitomycin C, mitotane, mitoxantrone, mitoguazone, nandrolone phenpropionate, Nofetumomab, nitrogen mustard, oprelvekin, oxaliplatin, paclitaxel, pamidronate, pegademase, pegaspargase, pegfilgrastim, pentostatin, pipobroman, plicamycin, porfimer sodium, procarbazine, progestins, prednimustine, PCNU, quinacrine, rasburicase, rituximab, sargramostim, streptozocin, talc, tamoxifen, temozolomide, teniposide (VM-26), testolactone, thioguanine, thiotepa, topotecan, toremifene, tositumomab, trastuzumab, tertinoin, uracil mustard, valrubicin, vinblastine, vincristine, vindesine, vinorelbine, and zoledronate.

Small molecule compounds that may be identified in the screening assay described above may include, but are not limited to, an acetanilide, aminoacridine, aminoquinoline, anilide, anthracycline antibiotic, antiestrogen, azalide, benzazepine, benzhydryl compound, benzodiazapine, benzofuran, beta-lactam, cannabinoid, cephalosporine, colchicine, cyclic peptide, dibenzazepine, digitalis glycoside, dihydropyridine, epipodophyllotoxin, ergot alkaloid, fluoroquinolone, imidazole, isoquinoline, lincosamide, macrolide, naphthalene, nitrogen mustard, opioid, oxazine, oxazole, phenothiazine, phenylalkylamine, phenylpiperidine, piperazine, piperidine, polycyclic aromatic hydrocarbon, pyridine, pyridone, pyrimidine, pyrrolidine, pyrrolidinone, quinazoline, quinoline, quinone, rauwolfia alkaloid, retinoid, rifamycin (ansamacrolide), salicylate, steroid, stilbene, sulfone, sulfonamide, sulfonylurea, taxol, tetracycline, triazole, tropane, or vinca alkaloid.

A compound whose activity is recognized by the invention can be formulated by any method known to one of ordinary skill in the art.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

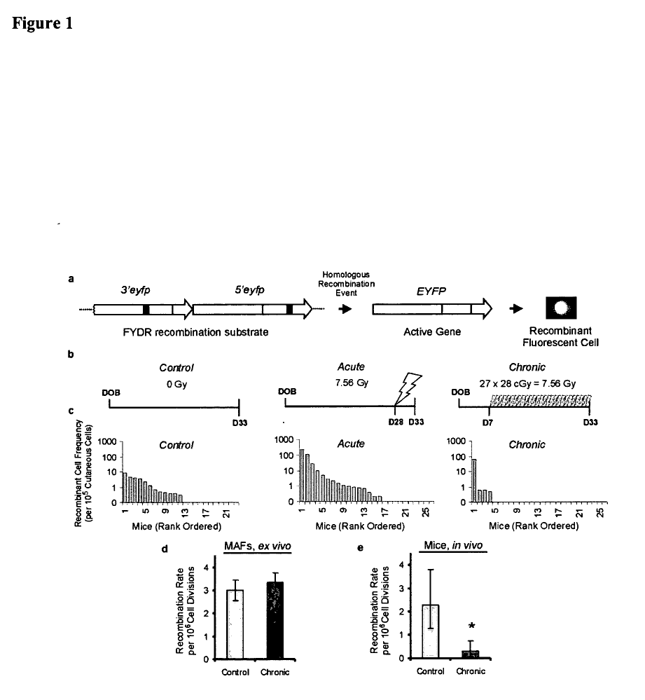

Ionizing radiation induces mutations and chromosomal rearrangements that can lead to cancer (Ron, E. (1998) Radiat. Res. 150:S30-S41). DNA rearrangements are caused by incorrect joining of double strand breaks (DSBs) by non-homologous end-joining (NHEJ), and by DSB-induced homologous recombination (Liang et al., (1998) Proc. Natl. Acad. Sci. USA, 95:5172-5177; Rothkamm et al. (2001) Cancer Res, 61:3886-3893; Jackson (2002) Carcinogenesis, 23:687-696). We recently created transgenic FYDR mice in which homologous recombination between two different truncated eyfp cDNAs can reconstitute full length coding sequence and cause cells to fluoresce in vivo (FIG. 1a) (Hendricks et al. (2003) Proc. Natl. Acad. Sci. USA, 100:6325-6330). Using flow cytometry, the recombinant cell frequency was measured in disaggregated cutaneous cells from 23 unexposed FYDR mice (FIG. 1b-c). As expected, the frequency of recombinant cells varied among individual mice (Hendricks et al. (2003) Proc. Natl. Acad. Sci. USA, 100:6325-6330), which reflects the probability of a recombination event occurring at different times during growth (Luria et al. (1943) Genetics, 28:491-511) (the frequency of fluorescent recombinant cells was below the limits of detection for about half of the mice).

We measured the effects of a single acute dose of ionizing radiation on recombination frequency in vivo (FIG. 1b). Exposure to 7.5 Gy (˜LD50 at 30 days post-irradiation) significantly increased the frequencies of recombinant cells (FIG. 1c; p≦0.05, Mann-Whitney test). The average frequency increased from 1.1 to 15.1 per 10 (Jackson, (2002) Carcinogenesis, 23:687-696) cells, which is consistent with previous studies showing that ionizing radiation induces homologous recombination in cultured cells and in mouse embryos (Aubrecht et al. (1995) Carcinogensis, 16:2841-2846; Benjamin and Little (1992) Mol. Cell. Biol., 12:2730-2738; Schiestl et al. (1997) Proc. Natl. Acad. Sci. USA, 94:4576-4581). Although X-rays induce potentially recombinogenic DSBs, most of these DSBs are rapidly repaired by NHEJ (Jackson (2002) Carcinogenesis, 23:687-696; Sargent et al. (1997) Mol. Cell. Biol., 17:267-277). Some DSBs may nevertheless induce homologous recombination if they are formed during S phase, when homology directed repair is most active (Rothkamm et al. (2003) Mol. Cell. Biol., 23:5706-5715; Haber (1999) Trends Biochem. Sci., 24:271-275). Interestingly, although the acute exposure induced recombination in some mice, for several mice, the recombinant cell frequencies were not greater than those of the control animals. One possible explanation for this apparent inter-mouse variability is that the proportion of cells in S-phase can be highly variable (even among mice within the same litter), depending on the state of fur regeneration (Potten et al. (1971) Cell Tissue Kinet., 4:241-254).

In addition to DSBs, X-rays also increase the levels of oxidized bases (Friedberg et al. (1995) DNA Repair and Mutagenesis, ASM Press, Washington, D.C.). These damaged bases are rapidly removed by DNA glycosylases to yield abasic (AP) residues that are subsequently repaired by downstream base excision repair (BER) enzymes (Friedberg et al. (1995) DNA Repair and Mutagenesis, ASM Press, Washington, D.C.). It is well established that BER intermediates, such as AP sites and single strand breaks, are recombinogenic during S phase (Swanson et al. (1999) Mol. Cell. Biol., 19:2929-2935; Memisoglu and Samson (2000) J. Bacteriol. 182:2104-2112; Sobol et al. (2003) J. Biol. Chem., 278:39951-39959). Given that not all of the cells in cutaneous tissue are in S phase at the time of the acute exposure (Potten et al. (1971) Cell Tissue Kinet., 4:241-254), we anticipated that daily exposure might increase the number of cells exposed to recombinogenic lesions during S phase. Thus, we anticipated that chronic irradiation would increase the levels of radiation-induced homologous recombination.

FYDR mice were exposed to 28 cGy daily until they reached a cumulative dose of 7.56 Gy (FIG. 1b). There were no overt signs of toxicity nor any significant effects on cell proliferation (as studied by Ki67 immunohistochemistry; data not shown). Rather than inducing homologous recombination, chronic irradiation reduced the frequency of recombinant cells to levels that are significantly below those of control animals (p<0.05, Mann-Whitney test) (FIG. 1c). One possible explanation for these observations is that chronic irradiation suppresses expression of EYFP, rendering recombinant cells undetectable by flow cytometry. Another possibility is that chronic irradiation suppresses the rate of recombination.

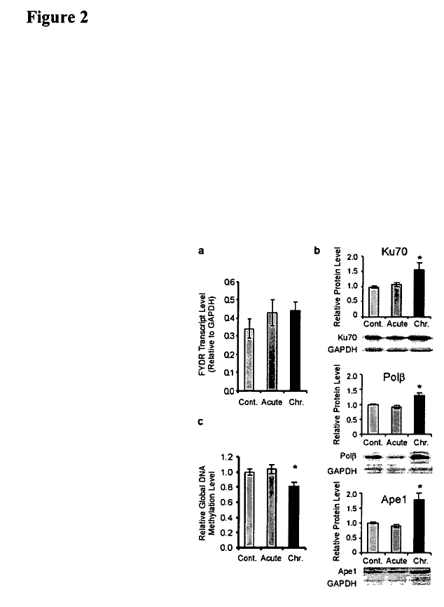

To determine if the recombination substrate had been inactivated by chronic irradiation, the recombination rate was evaluated in primary mouse adult fibroblasts (MAFs) derived from chronically irradiated mice. MAFs cultured from mice in the chronic cohort were not significantly different from control MAFs in their ability to give rise to recombinant fluorescent cells ex vivo (FIG. 1d). Thus, the recombination substrate could not have been permanently inactivated. However, it remained possible that EYFP expression was silenced in vivo by chronic irradiation, and that silencing had been reversed in culture. Therefore, we measured the transcript levels expressed from the FYDR recombination substrate in cutaneous tissues from mice in the control and irradiated cohorts. Radiation did not significantly affect transcription from this locus (FIG. 2a), suggesting that the reduced appearance of fluorescent recombinant cells in the chronically irradiated mice is not due to suppression of gene expression, but instead is due to suppression of homologous recombination.

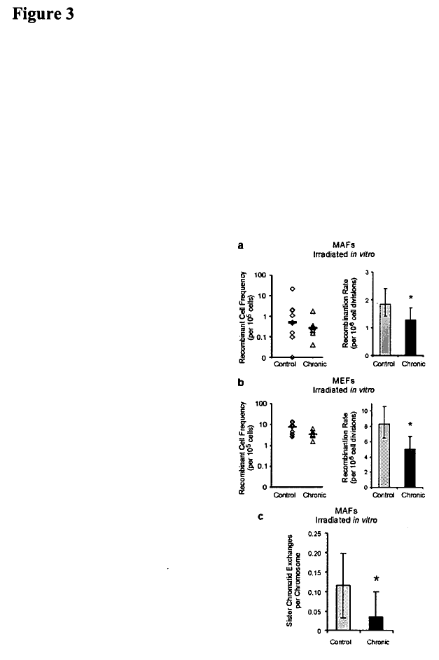

Using the data presented in FIG. 1c, we calculated the recombination rates in control and chronically irradiated mice and found that chronic irradiation significantly suppressed the recombination rate in vivo by ˜7 fold (FIG. 1e). In contrast, we had observed that the recombination rate in MAFs cultured from ear tissue of control and chronically irradiated mice were similar (FIG. 1d). Two possible explanations for this discrepancy are 1) the in vitro conditions do not accurately reflect the conditions in vivo; and 2) the suppressive effects of chronic irradiation on recombination are transient (e.g., the recombination rate had reverted to normal during the time that the cells were expanded for rate analysis in culture). To determine if chronic irradiation can suppress recombination in cultured cells, MAF cultures were created from independent FYDR mice and chronically irradiated during expansion in culture. Consistent with the effects of chronic exposure in vivo, chronic irradiation of cultured cells significantly suppressed the recombinant cell frequency (p<0.05, Mann-Whitney test) and the rate of homologous recombination (FIG. 3a). (Note that there were no overt signs of cytotoxicity, and the population doubling time was not affected by chronic irradiation [data not shown].) Despite the fact that the spontaneous recombination rate in FYDR mouse embryonic fibroblasts (MEFs) is significantly higher than in MAFs (Hendricks, C. A. (2003) Proc. Natl. Acad. Sci. USA, 100:6325-6330), chronic irradiation also suppressed homologous recombination in FYDR MEFs (FIG. 3b). Together with in vivo data, these results clearly show that chronic irradiation suppresses homologous recombination at the FYDR recombination substrate. To determine if there is genome-wide suppression of recombination, in addition to suppression specifically at the FYDR locus, we measured the frequency of sister chromatid exchanges (SCEs) in metaphase spreads. Strikingly, four daily exposures to 28 cGy significantly suppressed the frequency of SCEs in MAFs (FIG. 3c). Thus, chronic irradiation causes a general reduction in the frequency and rate of homologous recombination events, both in vivo and in vitro.

In 1977, Samson and Cairns discovered that E. coli exposed to non-toxic levels of an alkylating agent undergo an adaptive response and become resistant to subsequent toxic doses of the same agent (Samson and Cairns (1977) Nature, 267:281-283). In addition to chemicals, low doses of ionizing radiation also induce an adaptive response not only in prokaryotes, but also in mammals (Wolff (1998) Environ. Health Perspect, 106 Suppl 1:277-283). Adapted cells have increased defenses against DNA damage and have been shown to repair DSBs more rapidly (Ikushima et a 1. (1996) Mutation Res., 358:193-198). We therefore measured the levels of Ku70, a protein that is essential for NHEJ, in cutaneous samples from mice in the control, acute and chronically irradiated cohorts. Although we did not detect any significant induction of Ku70 in animals exposed to acute irradiation, there was a significant increase in the levels of Ku70 within tissue from chronically irradiated animals (FIG. 2b). Thus, enhanced NHEJ may help prevent DSB-induced homologous recombination.

Altered levels of BER enzymes can cause imbalanced BER, which leads to increased levels of recombinogenic BER intermediates (Swanson et al. (1999) Mol. Cell. Biol., 19:2929-2935; Memisoglu and Samson. (2000) J. Bacteriol., 182:2104-2112). Furthermore, DNA glycosylases can convert clustered base lesions into recombinogenic double strand breaks (Blaisdell et al. (2001) Proc. Natl. Acad. Sci. USA, 98:7426-7430). However, if BER enzymes are induced in a coordinated fashion, then recombinogenic BER intermediates would be cleared more efficiently. In mammals, AP endonuclease (Ape1) and polymerase β (Polβ) are critical for creating an extendable 3′OH group and a ligatable 5′ phosphate during BER (Friedberg et al. (1995) DNA Repair and Mutagenesis, ASM Press, Washington, D.C.). While a single acute exposure did not induce Polβ or Ape1, chronic irradiation clearly caused a significant increase in the levels of both these proteins (FIG. 2b). In addition to being formed by exposure to irradiation, oxidative base lesions and DSBs are also formed spontaneously. Thus, increased levels of NHEJ and enhanced clearance of BER intermediates may help suppress both radiation-induced recombination and spontaneous homologous recombination events in the chronically irradiated animals.

Gene silencing is often controlled by methylation at CpG islands, and it is known that DNA damage can alter methylation patterns (Wilson and Jones (1983) Cell 32:239-246; Kovalchuk et al. (2004) Mutation Res., 548:75-84). Here, we show that chronic irradiation reduces the levels of global methylation in the cutaneous tissue of mice (FIG. 2c). Although the levels of only three proteins were evaluated by Western, this shift in global methylation suggests a widespread shift in gene expression patterns. Thus, in addition to Ku70, Polβ and Ape1, it is likely that there are other genes that impinge on cellular susceptibility to homologous recombination.

Radiation is one of the most broadly effective agents used in cancer therapy. Typically, patients receive daily doses of 120-300 cGy per day, five days a week for several weeks (Connell et al. (2004) DNA Repair in press). Here, we have shown that daily exposure to 28 cGy induces an adaptive response that alters the spectrum of DNA damage and repair responses in vivo. Similar changes in gene expression may also be induced in patients who are exposed to therapeutic radiation, which could affect their prognosis. For example, the efficacy of radiation therapy may depend on the ability of the tumor to undergo an adaptive response. Indeed, it has been shown that several transformed cell lines are unable to undergo an adaptive response and are thus sensitized to radiation-induced apoptosis (Park et al. (1999) Cell. Biol. Toxicol., 15:111-119), whereas radioresistant human gliomas undergo a robust adaptive response (Smith et al. (2003) Int. J. Radiat. Biol., 79:333-339. Thus, at least in some cases, knowledge about the relative sensitivity of the patient versus the tumor specifically under adaptive conditions might guide optimization of radiotherapy for individual patients.

Methods

Irradiation of animals. Mice heterozygous for the FYDR recombination substrate were randomly assigned to different treatment groups. Animals were housed in a virus-free facility and given food and water ad libidum. The ‘chronic’ group received 28 cGy (2cGy/sec) whole body X-rays applied daily for 27 consecutive days until reaching a total dose of 7.56 Gy. The ‘acute’ group received a single dose of 7.56 Gy (2cGy/sec) on day 28. Control mice were sham treated. All animals were humanely sacrificed at the age of 33 days upon completion of the treatment protocol. Cutaneous tissue was isolated immediately upon sacrifice, and processed for subsequent molecular studies (see below), fixed in 4% paraformaldehyde for immunohistochemical staining, or disaggregated for analysis of recombinant cell frequency. Concomitantly, cells were isolated from ear tissue of 4 mice from each cohort and cultured ex vivo to determine the recombination rate ex vivo.

Estimates of recombination frequencies and rates. The frequency of recombinant cells in disaggregated cutaneous tissues was measured by flow cytometry as previously described (Hendricks (2003) Proc. Natl. A cad. Sci. USA, 100:6325-6330). The in vivo recombination rates were calculated using the p₀ method (Luria et al. (1943) Genetics, 28:491-511), under the assumption that the number of cell divisions can be approximated by the number of cells analyzed by flow cytometry.

MAFs were isolated from ear tissue of unexposed and irradiated FYDR mice and expanded in culture for 3 days. Cells were then plated into 22-24 independent cultures and expanded an additional 5-7 days. Recombinant cell frequencies were measured by flow cytometry and the recombination rate was calculated using the p₀ method (Luria et al. (1943) Genetics, 28:491-511).

For in vitro irradiation studies, MAFs and MEFs were isolated from unexposed FYDR mice and embryos, respectively. MAF cultures were derived from independent mice, whereas MEF cultures were created from a single homogenous population of cells. The frequency of recombination among 9-10 independent MAF and MEF cultures was determined by flow cytometry after 6 daily exposures to 28 cGy using a Cs-137 Gamma Cell G40 (63 rads/min). The recombination rates were calculated using the MSS Maximum Likelihood Method (Rosche et al. (2000) Methods, 20:4-17). For SCEs, cultured MAFs were exposed to 28 cGy for four days (or sham treated), and BrdU was added to the media 6 h after the last exposure. After 48 h, colcemid was added and cells were incubated for an additional 14 hours prior to isolation of mitotically arrested cells. SCEs were stained as previously described (Sobol et al. (2003) J. Biol. Chem., 278:39951-39959) and counted in a blinded fashion.

RNA preparation, reverse transcription and real-time PCR. For RNA preparation, cutaneous tissues were sampled upon sacrifice and immediately equilibrated in RNAlater solution (Qiagen), according to the manufacturer's instructions. Total RNA was isolated using Trizol reagent (Life Technologies). RNA samples were treated with DNAse I (Invitrogen) and RNA was further purified using the RNeasy total RNA cleanup protocol (Qiagen). The RNA yields were measured using RiboGreen assay (Molecular Probes). Reverse transcription was performed using RevertAid™ H Minus First Strand cDNA Synthesis Kit (Fermentas). Primers that amplify a 112 bp segment of coding sequence that is present in both the unrecombined FYDR substrate and in full length EYFP were used to evaluate the levels of transcripts expressed from the FYDR locus.

Real-time PCR was performed in a total volume of 25 μL using 1 μL of the 1^{st} strand cDNA synthesis mixture as a template, 300 nM of primers and 12.5 μL of 2×SYBRGreen PCR Master Mix (Applied Biosystems). Duplicate reactions were carried out with 1:3 and 1:15 dilutions of the 1^{st} strand cDNA synthesis mixture. A SmartCycler (Cepheid, Sunnyvale, Calif.) was used to perform PCR and fluorescence was quantified against standards. Wells containing SYBR Green PCR master mix and primers without sample cDNA were used as negative controls and emitted no fluorescence. Levels of FYDR transcripts were standardized against glyceralaldehyde-3-phosphate dehydrogenase (GAPDH; levels were measured in parallel). Primer sequences are available upon request.

Quantification of global DNA methylation. Total DNA was prepared from cutaneous tissues using Qiagen DNeasy kit (Qiagen). A cytosine extension assay was performed to measure the relative levels of DNA methylation (Kovalchuk et al., (2004) Mutation Res. 548:75-84).

Western immunobloting. Cutaneous tissue was snap frozen immediately after isolation. Protein samples were sonicated in 1% sodium dodecyl sulphate and boiled for 10 min. Proteins were separated by SDS-PAGE and transferred to PVDF membranes. Membranes were incubated with antibodies against Ku70 (BD Biosciences), Ape1/Ref1 (Biomira), Polβ (Novus Biologicals), and GAPDH (Santa Cruz). Antibody binding was revealed by incubation with HRP-conjugated secondary antibodies (Amersham) and the ECL Plus detection system (Amersham). PVDF membranes were stained with Coomassie Blue (BioRad) and the intensity of the Mr 50,000 protein band was assessed as a loading control. Signals were quantified using NIH Image 1.63 Software and normalized relative to both GAPDH and to the Mr 50,000 protein band, which gave internally consistent results (values relative to Mr 50,000 are plotted).

Statistical Analysis. Statistical analysis was performed using MS Excel 2000, Analyze-It, JMP5, and Mathematica software packages.

### Example 2

We have created several fluorescence-based recombination assays. By site-specifically integrating a matched pair of recombination substrates, we can delineate the full spectrum of classes of recombination. Using this approach, it is possible differentiate between single strand annealing and other classes of non-conservative recombination events (such as unequal sister chromatid exchanges). We have shown that single strand annealing is a common spontaneous recombination event in mammalian cells.

In addition, we created an animal model that makes it possible to directly detect recombinant cells in multiple mouse tissues by a fluorescent signal. We developed rigorous quantitative assays to measure the rate of recombination in cultured cells and in animals. We have used the engineered mice to reveal unexpected effects of chronic damage exposure, and have shown that recombinant cells can be observed within intact pancreatic tissue. We have also found that recombinant cells are readily detectable in pancreatic tissue of these transgenic mice.

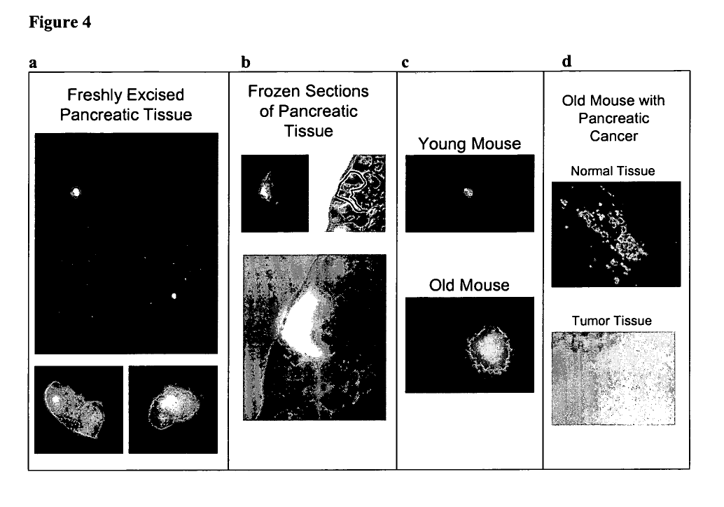

In particular, we observed that fluorescent pancreatic cells can be seen in freshly isolated tissue from FYDR mice. In young animals we often observed single cells as well as small groups of cells that likely result from clonal expansion of a recombinant cell (FIG. 4A). Serial sections revealed which cells carry recombinant DNA (FIG. 4B).

Further analysis comparing young and old mice revealed that recombinant cells clonally expanded during aging. As shown in FIG. 4C, the average fluorescent cluster size increases with age, raising the possibility that stem cells have undergone homologous recombination.

Pancreatic cancer may be associated with an abnormal distribution of recombinant cells. We have identified a mouse that had a spontaneous pancreatic tumor and we are examining the relationship between recombinant cells and cancer in vivo. We observed that most of the cells within the tumor were fluorescent (FIG. 4D). Furthermore, we observed that the pattern, frequency and distribution of recombinant cells is abnormal throughout the pancreatic tissue, resulting in large regions of pancreatic tissue being dotted with fluorescent cells (FIG. 4D).

These results suggest that normal pancreatic tissue in an animal suffering from pancreatic cancer undergoes widespread changes in patterns of proliferation and silencing. Alternatively, pancreatic cancer may be associated with hyper-recombination. If pancreatic cancer cells have an increased capacity to perform homology directed repair, then this result may help to explain why pancreatic cancers are among the least treatable cancers among humans, i.e., approximately 90% of people diagnosed with pancreatic cancer die within one year. Hyper-recombination may not only promote cancer, but may also render resulting tumor cells highly resistant to toxicity associated with standard chemotherapeutic agents, such as chemicals that introduce interstrand crosslinks. Thus, the very feature that renders tumor cells able to rapidly evolve may also make them resistant to DNA damaging agents.

Although recombinant cells can be visualized in situ within pancreatic tissue, the locus of integration for the recombination substrate prevents expression in several other tissues of interest, such as brain and colon. Consequently, we have created targeted embryonic stem cells in which an analogous recombination reporter is integrated into a locus compatible with detection in a broader set of tissues.

### Incorporation by Reference

All of the U.S. patents and U.S. patent application publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method for determining a substantially optimal dose of radiation needed to inhibit tumor growth, comprising the steps of administering a course of radiation therapy to a subject prior to surgery to remove a tumor; removing the tumor by surgery; dividing the tumor into a plurality of samples; exposing independently the plurality of samples to subsequent doses of radiation; and monitoring the plurality of samples for adaptive responses.

2. The method of claim 1, wherein the subject is exposed to a total dose of radiation from about 5 to 15 Gy prior to surgery to remove a tumor.

3. The method of claim 2, wherein the total dose of radiation is administered in five doses.

4. The method of claim 3, wherein each dose of radiation is about 1 to 3 Gy.

5. The method of claim 2, wherein the source of radiation is selected from the group consisting of x-ray radiation, gamma-ray radiation, UV radiation, microwaves, electronic emissions, and particulate radiation.

6. The method of claim 2, wherein the source of radiation is x-ray radiation.

7. The method of claim 1, further comprising obtaining a healthy tissue sample from a subject during surgery to remove a tumor mass, and monitoring said healthy tissue for an adaptive response.

8. The method of claim 1, wherein the sample is exposed to subsequent doses of radiation varying from about 0.5 to about 4 Gy.

9. The method of claim 1, wherein the sample is exposed to four or five doses of radiation.

10. The method of claim 1, wherein the source of radiation for subsequent doses of radiation is selected from the group consisting of x-ray radiation, gamma-ray radiation, UV radiation, microwaves, electronic emissions, and particulate radiation.

11. The method of claim 1, wherein the source of radiation is x-ray radiation.

12. The method of claim 1, wherein the adaptive response is monitored by measuring the expression of γ-H2A expression.

13. The method of claim 1, wherein an adaptive response is monitored by measuring cell survival.

14. A method for determining a substantially optimal dose of radiation needed to inhibit tumor growth, comprising the steps of obtaining a tumor tissue sample from a subject; exposing the tumor tissue sample to varying doses of radiation ex vivo; and monitoring the adaptive response of the tumor tissue sample.

15. The method of claim 14, further comprising obtaining a healthy tissue sample from a subject; exposing said healthy tissue sample to radiation ex vivo; and monitoring said healthy tissue for an adaptive response.

16. The method of claim 14, wherein the tissue sample is obtained from a subject during a biopsy procedure.

17. The method of claim 14, wherein the tissue sample is obtained from a subject during surgery.

18. The method of claim 14, wherein the tissue sample is exposed to varying doses of radiation range from about 0.5 to about 4 Gy.

19. The method of claim 14, wherein the tissue sample is exposed to four or five doses of radiation.

20. The method of claim 14, wherein the source of radiation is selected from the group consisting of x-ray radiation, gamma-ray radiation, UV-irradiation, microwaves, electronic emissions, and particulate radiation.

21. The method of claim 14, wherein the source of radiation is x-ray radiation.

22. The method of claim 14, wherein the adaptive response is monitored by measuring γ-H2A expression.

23. The method of claim 14, wherein an adaptive response is monitored by measuring cell survival.

24. A method for identifying chemotherapeutic drugs that are effective during and after radiation therapy, comprising the steps of pre-adapting target cells to radiation; screening the pre-adapted target cells against a plurality of small molecule compounds; and identifying small molecule compounds that induce DNA damage in the pre-adapted target cells.

25. The method of claim 24, wherein the target cells are pre-adapted to radiation following exposure to about 1 to about 3 Gy of radiation.

26. The method of claim 24, wherein the target cells are pre-adapted to radiation following exposure to about four or about five doses of radiation.

27. The method of claim 24, wherein the source of radiation to pre-adapt the target cells is selected from the group consisting of x-rays, gamma-rays, UV-irradiation, microwaves, electronic emissions, and particulate radiation.

28. The method of claim 24, wherein the source of radiation to pre-adapt the target cells is x-ray radiation.

29. The method of claim 24, wherein small molecule compounds that induce DNA damage in the pre-adapted target cells are identified by monitoring cell survival.

30. The method of claim 24, wherein small molecule compounds that induce DNA damage in the pre-treated target cells are identified by monitoring the induction of cell survival.

31. The method of claim 24, wherein small molecule compounds that induce DNA damage in the pre-adapted target cells are identified by detecting the expression of γ-H2A.
